(19) United States
(12) Patent Application Publication
(10) Pub. No.: US 2020/0315731 A1
(43) Pub. Date: Oct. 8, 2020

(54) DEVICE TO SUPPORT AT LEAST ONE ARTICLE TO BE SUBJECTED TO WASHING, STERILIZATION AND/OR HEAT DISINFECTION TREATMENT

(71) Applicant: STEELCO S.P.A., Riese Pio X (IT)

(72) Inventors: Fabio Zardini, Castelfrance Veneto (IT); Ivone Capovilla, Loria (IT)

(73) Assignee: STEELCO S.P.A., Riese Pio X (IT)

(21) Appl. No.: 16/304,247

(22) PCT Filed: May 23, 2017
(86) PCT No.: PCT/EP2017/062378

§ 371 Date: Nov. 23, 2018

(30) Foreign Application Priority Data
May 23, 2016 (IT) 102016000052863

(51) Int. Cl. A61B 50/22; A61L 2/04; A61L 2/26; A61B 1/12; B08B 9/023

(52) U.S. Cl. CPC A61B 50/22; A61L 2/04; A61L 2/26; A61L 2202/24; B08B 9/023; A61L 2202/17; A61B 1/121

(57) ABSTRACT

A device to support an article with an oblong and flexible configuration, to be subjected to a washing, sterilization and/or heat disinfection treatment, including a frame defining a support plane configured to receive and support one article to be treated. The support device includes a plurality of holding and positioning elements associated with the frame and that define a plurality of housing seatings disposed aligned along a positioning path and each configured to receive and hold a portion of the article in a predefined position.

### FIELD OF THE INVENTION

Embodiments described here concern a device to support articles, also called rack, used to support at least one article to be subjected to treatment in a suitable treatment machine, for example for washing, sterilization and/or heat disinfection.

In particular, the support device allows to load, position and support an article in the treatment steps, and also during the transport steps, these including the steps of supplying/selling the articles to clients.

The support device according to some embodiments described here can be used, although not exclusively, for articles of the medical type, surgical instruments, diagnostic investigation instruments, such as endoscopes, laboratory instruments or in general instruments that need frequent treatments, for example washing, sterilization or heat disinfection and subsequent drying.

### BACKGROUND OF THE INVENTION

It is known that there is a need to treat articles, usually medical articles, such as endoscopes, in suitable treatment machines, for example to wash, dry, sterilize and disinfect them.

To this purpose, support devices are known, usually called racks in the particular field, in which the articles are placed in order to be supported during transport, loading and treatment in the treatment machine.

The racks usually have a reticular configuration to allow washing jets to pass between the meshes, to reach the article to be treated, and to prevent the stagnation of washing liquids.

Known racks usually have a tray shape, that is, they are defined by a flat part with peripheral containing edges with the function of containing the article laterally.

The articles are usually disposed in the rack loose, without excluding the fact that several parts of the article can overlap. The overlap between parts of the articles does not allow the washing or disinfection jets to efficiently reach the entire article, thus precluding effective treatment.

A further disadvantage of known racks is that, because of the high pressures of the washing, and/or drying, and/or disinfection jets, the article is displaced or moved inside the rack. This displacement can carry the article, or part of it, even outside the washing rack, where it can interfere with operating parts of the treatment machine.

It is also known that at least some types of articles, for example endoscopes, are provided not only with operating terminals but also with connection elements, such as cables and/or pipes, which are connected solidly with the operating terminals with a length of even more than two meters.

The connection element, when inserted loose into the rack, can not only overlap, causing the above-mentioned problems, but can also get knotted and/or tangled, making the removal of the article from the treatment machine complex and precluding, at times, the effectiveness of the treatment performed.

EP-A-2.025.282 describes a rack for containing and supporting an endoscope during the washing steps. The rack is defined by a shaped net having a support wall for the endoscope and a pair of containing walls protruding from the support wall and defining a containing seating to support the endoscope.

Some clamping elements are attached on the support wall provided to attach only terminal parts of the endoscope such as a main connector, a peripheral connector, and ends of insertion pipes.

However, the solution described in this document has the disadvantage that the flexible parts of the endoscope, that is, the flexible connection element between its terminal parts, are not constrained to the rack and can move during transportation, washing and handling, causing the problems described above.

Furthermore, due to the shape of the net, this solution is particularly cumbersome both for the washing steps and the transport steps.

There is therefore a need to perfect a device to support articles that can overcome at least one of the disadvantages of the state of the art.

In particular, one purpose of the present invention is to provide a device to support articles that increases the efficiency of the treatment of the article itself.

Another purpose of the present invention is to provide a device to support articles that facilitates operations at least to remove the article after treatment.

Another purpose of the present invention is to provide a support device to be used to support articles not only during the treatment steps.

Other limitations and disadvantages of conventional solutions and technologies will be clear to a person of skill after reading the remaining part of the present description with reference to the drawings and the description of the embodiments that follow, although it is clear that the description of the state of the art connected to the present description must not be considered an admission that what is described here is already known from the state of the prior art.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purposes, embodiments described here concern a support device configured to support an article during a washing, sterilization and/or heat disinfection treatment, the article having an oblong and flexible configuration. The article is provided with a first terminal part, at least a second terminal part and a flexible connection element connected between the first terminal part and the second terminal part.

In accordance with possible embodiments described here, the support device comprises:
a frame configured to receive and support the article to be treated,
a first positioning mean attached to the frame and configured to support and hold the first terminal part and—a second positioning mean attached to the frame and configured to support and hold the second terminal part.

In accordance with possible embodiments, the support device comprises a plurality of holding and positioning elements associated with the frame and defining a plurality of housing seatings disposed aligned along a positioning path. The housing seatings are also configured to receive and hold a portion of the flexible connection element in a predefined position.

This solution allows to associate the article to be treated with the frame defining a predefined and stable position of the latter with respect to the support plane.

In this way, during the treatment operations the article does not exit from the support device, so it does not interfere with moving parts of the treatment machine and is not subjected to movements that could cause overlapping between parts of the article, which would cause an ineffective treatment.

Embodiments described here also concern a combination of at least one article and a support device as described here.

Other forms of embodiment of the present invention also concern a method for the washing, sterilization and/or heat disinfection of at least one article with a flexible, oblong configuration, and provided with a first terminal part, at least a second terminal part and a flexible connection element connected between the first terminal part and the second terminal part. The method comprises at least the disposal of an article on a frame and the support and holding of the first terminal part and of the second terminal part in a first positioning mean and respectively in a second positioning mean.

In accordance with possible implementations, the method comprises the insertion and holding of portions of the flexible connection element in housing seatings disposed aligned along a positioning path and defined by a plurality of holding elements associated with the frame.

These and other aspects, characteristics and advantages of the present disclosure will be better understood with reference to the following description, drawings and attached claims. The drawings, which are integrated and form part of the present description, show some forms of embodiment of the present invention, and together with the description, are intended to describe the principles of the disclosure.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example with reference to the attached drawings wherein:

FIG. 1 is a perspective view of a support device for articles, in accordance with a possible solution;

FIG. 2 is a plan view of a support device with which an article is associated;

FIGS. 3-5 are views of enlarged details, according to possible variants, of parts of a support device for articles;

FIG. 6 shows a possible variant of FIG. 2;

FIG. 7 is an enlarged detail of FIG. 6;

FIG. 8 is a section view of a possible variant of FIG. 7.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We shall now refer in detail to the various embodiments of the present invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

Embodiments of the present invention concern a support device indicated in its entirety by the reference number 10, able to support at least one article 13 to be subjected to washing, sterilization and/or heat disinfection treatment.

The support device 10 comprises a frame 11 configured to receive and support the article 13.

In some embodiments of the invention, the frame 11 defines a support plane 12 on which to position the article 13 to be treated.

According to possible solutions of the invention, the frame 11 has a flat shape allowing in this way a high containment of the bulk of the support device 10 during both the transport steps and in the washing steps.

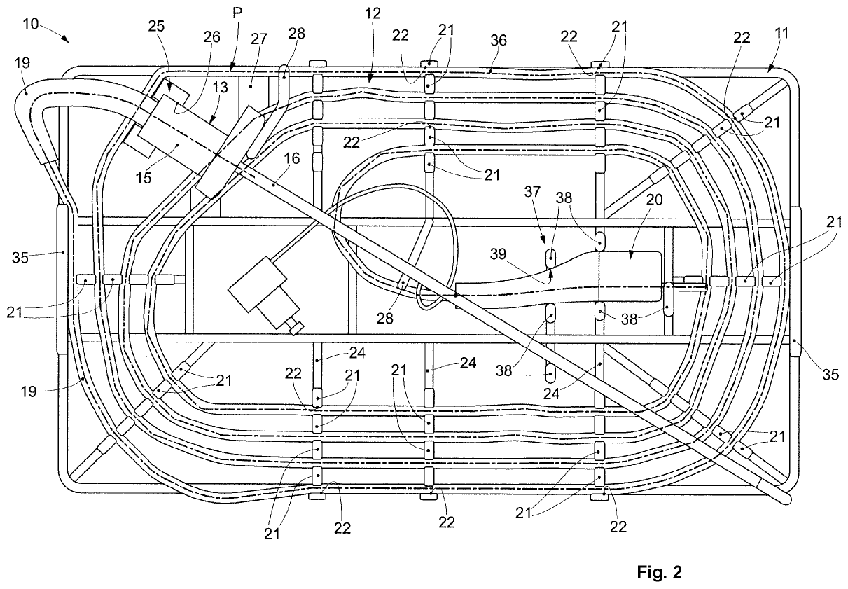

With reference to FIGS. 2, 6-8, the article 13 has an oblong shape and is flexible to adapt to different positioning configurations.

Although hereafter in the description we will refer to an endoscope 13 as the article, it is not excluded that the support device 10 can be used to support different articles.

With reference to FIGS. 2, 6-8, the endoscope 13 is provided with two terminal parts 14, 20.

In particular, a first terminal part or operating terminal 14 is provided, to which operative and/or optical vision devices are associated.

Furthermore, a second terminal part or connection terminal 20 is provided.

In particular, the operating terminal 14 can be provided with a manipulation body 15 and a tubular element 16 attached to the manipulation body 15.

The manipulation body 15 allows the operator to grip it, in order to insert the tubular element 16 into the parts of the organism to be analyzed.

The tubular element 16 can allow to deliver analysis liquids and, for this purpose, is fluidly connected to feed channels 17 provided in the operating terminal 14, in the case shown here in the manipulation body 15.

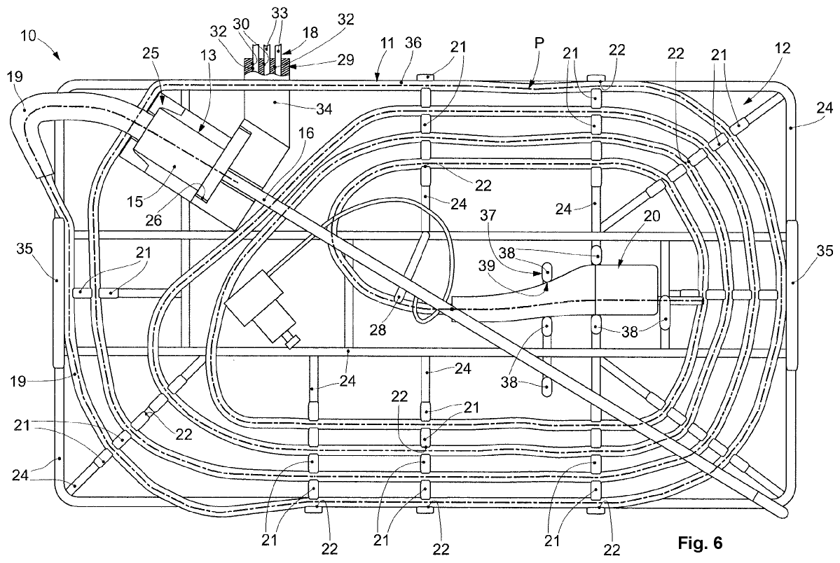

The tubular element 16 can be rigid, as in the solutions shown in FIGS. 2 and 6, or flexible.

The feed channels 17, during use, can be connected to devices to feed the analysis liquids, or, during the washing operations, can be connected to devices 18 to feed treatment liquids, for example in the manner described below.

The endoscope 13 is also provided with a flexible connection element 19 connected with a first end to the operating terminal 14 and with a second end to said connection terminal 20.

The connection element 19 can have a filiform configuration.

According to one embodiment, the connection element 19 can have a circular cross section.

The connection element 19 can comprise one or more cables and/or one or more pipes.

The cables can allow to transfer data and/or power supply between electronic command apparatuses, and apparatuses to receive and process data to operating and/or viewing devices.

The pipes, if provided, can allow to feed analysis liquids to the feed channels 17 of the operating terminal 14. In accordance with variant embodiments, the feed channels 17 extend along the connection element 19 to the connection terminal 20.

The connection terminal 20 therefore allows to electrically connect possible operational and/or optical vision devices provided in the operating terminal 14 with electronic apparatuses for data reception and processing and/or if provided with feed devices for analysis liquids.

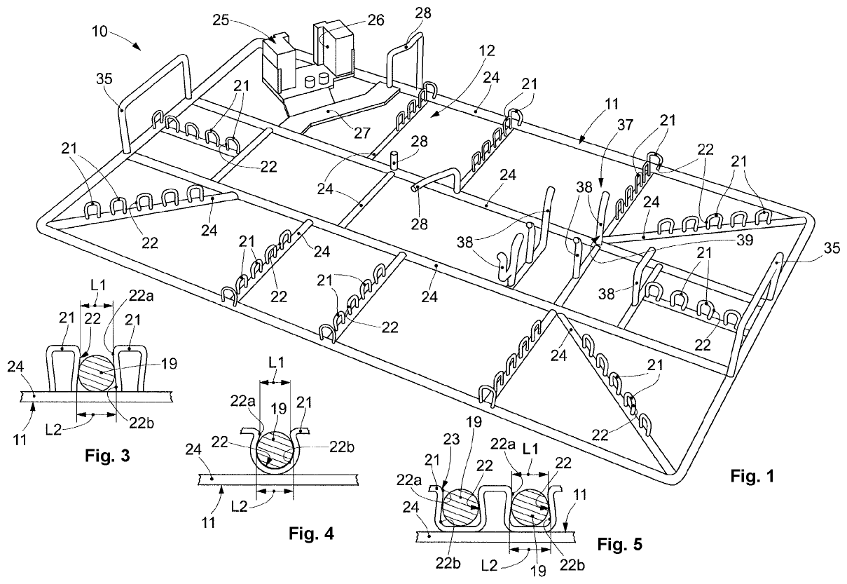

In the case shown in FIGS. 1 and 2, the frame 11 has a substantially rectangular shape although it is not excluded that in other embodiments the frame 11 can have a different shape, for example circular or polygonal.

In accordance with possible embodiments of the present invention, the support plane 12 of the frame 11 can be substantially flat, as in the solution shown in FIGS. 1, 2, and 6, or can have conformations with several offset or inclined planes, and defined according to particular application needs.

In possible embodiments, the frame 11 is a reticular frame, that is, configured in the form of a lattice or net, defined by meshes obtained from arms, bars or similar interconnected elements. This reticular conformation allows the passage of the fluids and/or treatment liquids and their drainage when necessary.

In accordance with possible solutions, the frame 11 can advantageously be made of material suitable for supporting conditions of washing, sterilization and/or heat disinfection treatments, for example with a metal material, for example steel, stainless steel or aluminum, or with a polymer material, for example, polyethylene or polyamide.

In accordance with one aspect of the present invention, the frame 11 is provided with a plurality of holding and positioning elements 21 associated with the frame 11, defining a plurality of housing seatings 22 disposed aligned along a positioning path P and configured to receive and hold a portion of the endoscope 13 in a predetermined position, that is, a portion of the flexible connection element.

The positioning path P can be defined by rectilinear segments and curved segments to dispose the endoscope 13 on the support plane 12 in an organized manner, substantially affecting the entire surface of the latter and preventing overlapping.

In accordance with possible solutions, the positioning path P can have a spiral conformation with a plurality of spirals that wind continuously one inside the other.

According to a possible solution, the holding and positioning elements 21 are made as separate elements and subsequently connected to the frame 11. In particular, the holding and positioning elements 21 can be connected to the frame 11 by mechanical connection and/or by welding.

In accordance with a variant embodiment, the holding and positioning elements 21 can be obtained in a single body with the frame 11, for example by molding.

According to possible solutions, the housing seatings 22 have an undercut conformation.

By undercut conformation, we mean that the housing seating 22 has an insertion portion 22a open toward the outside, and a housing portion 22b, which is internal to the insertion portion 22a, and in which a portion of the endoscope 13 is positioned.

The insertion portion 22a has smaller dimensions than those of the housing portion 22b, which in turn has a size and shape suitable to accommodate the portion of the endoscope 13.

Merely by way of example, the insertion portion 22a has a first width L1, while the housing portion 22b has a second width L2 less than the first width L1. The second width L2 substantially corresponds to the maximum width of the housing portion 22b.

The undercut conformation of the housing seating 22 allows to insert a portion of the endoscope 13 into the latter, exploiting the elasticity of the latter. This ensures the holding of the endoscope 13 to the frame 11 even when there are elastic return actions, for example of the electric cable 19, or the frame 11 is inclined, or upturned.

In accordance with possible embodiments shown, for example, in FIGS. 1-3 and 6, it is provided that at least two holding and positioning elements 21 are placed side by side with each other, on one side and the other with respect to a portion of the positioning path P, and together define said housing seating 22 configured to accommodate a portion of the article 13.

In accordance with the solution shown in FIGS. 1 and 3, the holding and positioning elements 21 are U-shaped, that is, they are provided with two segments facing each other and connected by an intermediate connection segment. The free ends of the facing segments are, in turn, connected to the frame 11.

In accordance with a possible variant embodiment, for example shown in FIG. 4, each holding and positioning element 21 itself defines a housing seating 22 in which to accommodate a portion of the endoscope 13.

According to a further variant embodiment, as shown in FIG. 5, a plurality of said holding and positioning elements 21 can be made in a single body, for example by shaping a shaped wire 23 defining said housing seatings 22.

In accordance with a possible solution, shown in FIGS. 1 and 2, the frame 11 is defined by a plurality of bars 24, integrally connected with each other to define said support plane 12.

At least some of the bars 24 can be bent/shaped to define, for example, the perimeter of the frame 11.

The bars 24 can be reciprocally connected to each other solidly, for example by welding.

In accordance with variant embodiments, not shown, the bars 24 can be reciprocally coupled in a removable manner to obtain a modular frame 11, with desired and predefined sizes on each occasion.

In accordance with the solutions shown in FIGS. 1-6, the holding and positioning elements 21 are attached to at least some of the bars 24.

In particular, it can be provided that groups of holding and positioning elements 21 are connected to the bars 24 aligned with each other in order to define, between adjacent pairs of holding and positioning elements 21, said housing seatings 22.

In accordance with a further solution, the support device 10 can be provided with a first positioning mean 25 attached to the frame 11 and configured to receive and keep the manipulation body 15 of the endoscope 13 in position.

The first positioning mean 25 can be provided with a positioning seating 26 in which the manipulation body 15 is located.

The positioning seating 26 can be open at the upper part to allow the manipulation body 15 to be inserted from above, and laterally to allow the tubular element 16 and the connection element 19 to be positioned through it.

According to a possible solution, the first positioning mean 25 is configured to position the endoscope 13 diagonally to the rectangular development of the support plane 12.

To this purpose, the first positioning mean 25 can be attached to the frame 11 in correspondence with a vertex thereof.

The frame 11 can be provided with a support bracket 27 on which the first positioning mean 25 can be installed.

According to possible solutions, the support device 10 comprises a second positioning mean 37 attached to the frame 11 and configured to support and hold at least the said second terminal part 20, that is, the connection terminal.

In accordance with a possible solution, the second positioning mean 37 can comprise a plurality of filiform brackets 38 attached to the frame 11 and suitably shaped to define a holding and containing cavity 39 for the connection terminal 20.

According to a possible variant embodiment, not shown, the second positioning mean 37 can be defined by a support body provided with a holding and containing cavity configured to accommodate the connection terminal 20.

According to a possible solution, it can be provided that the first positioning mean 25 defines a first end of said path P, the second positioning mean 37 defines a second terminal end of said path P while the holding and positioning elements 21 define intermediate segments of the path P contained between the first positioning mean 25 and the second positioning mean 37. In accordance with possible solutions, the frame 11 can also be provided with stabilizer elements 28 configured to stabilize the positioning of at least one of either the operating terminal 14 or the electric connection terminal 20 of the endoscope 13.

The stabilizer elements 28 can comprise shaped filiform bars configured to define at least one of the housing seatings and/or abutment portions and/or support portions for parts of the endoscope 13.

In accordance with possible variant embodiments of the present invention, the first positioning mean 25 can be provided with connection devices 29 which can be connected on one side to the devices to feed treatment liquids 18 and on the other side to the feed channels 17 provided in the manipulation body 15 of the endoscope 13.

The connection devices 29 can comprise at least one connection channel 30, in the case shown here a plurality of connection channels 30 associated with, or made or integrated in the first positioning mean 25.

In possible solutions, the connection channels 30 can be provided with respective first connection ends 31 which allow to connect the connection channels 30 to the feed channels 17.

The first connection ends 31 are positioned in a coordinated manner with the positioning of the feed channels 17 disposed in the manipulation body 15.

The first connection ends 31 can be positioned protruding from the positioning seating 26 of the first positioning mean 25. In this way, by inserting the manipulation body 15 into the positioning seating 26, the simultaneous connection is obtained of the first connection ends 31 with the feed channels 17.

Each connection channel 30 can further comprise at least a second connection end 32 opposite the first connection end 31 and provided to allow the fluidic connection between the connection channel 30 and the devices to feed treatment liquids 18.

In particular, it is provided that the second connection ends 32 are positioned in a predefined pattern and coordinated with the positioning of corresponding connection portions 33 of the devices to feed treatment liquids 18.

According to a possible solution, the first positioning mean 25 is provided with an appendix 34 in which the at least one second connection end 32 is provided.

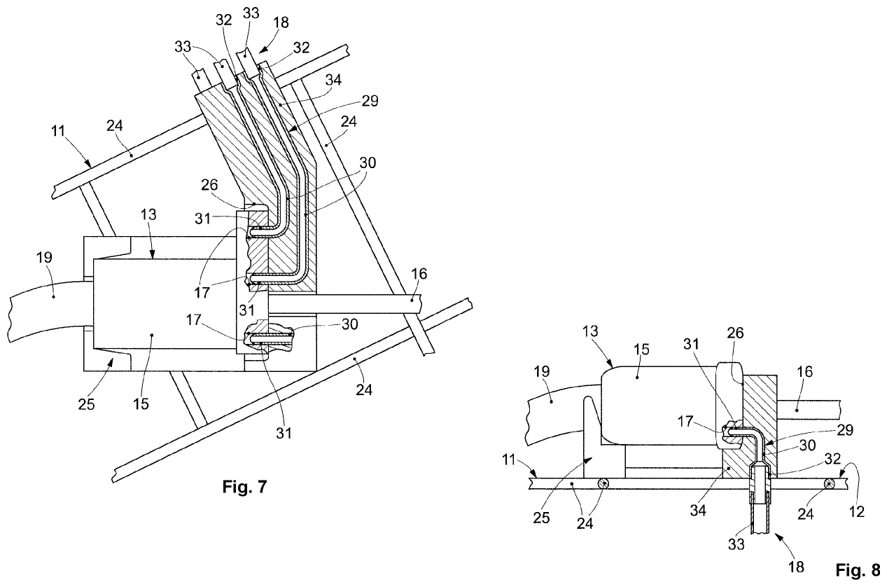

In accordance with a possible solution, shown in FIGS. 6 and 7, the appendix 34 is positioned in correspondence with a lateral edge of the frame 11.

The appendix 34 can be substantially flush with respect to the lateral edge or can protrude with respect therefrom.

This solution allows to obtain the reciprocal connection between the second connection ends 32 and the devices to feed treatment liquids 18 during or after insertion of the support device 10 into a treatment machine.

Merely by way of example, it can be provided that the connection portions 33 are disposed in the treatment machine in a predetermined position, coordinated with the position of the appendix 34.

By inserting and positioning the support device 10 in the treatment machine, the simultaneous connection is obtained of the connection portions 33 and the second connection ends 32.

According to a variant embodiment, after insertion of the support device 10 into the treatment machine, the devices to feed treatment liquids 18 are moved to connect to the connection portions 33.

With reference to FIG. 7, a further variant embodiment is shown in which the appendix 34 of the first positioning mean 25 faces toward the lower part of the support plane 12 allowing the subsequent connection of the devices to feed treatment liquids 18.

According to this solution too, it can be provided that the reciprocal connection between the devices to feed treatment liquids 18 and the second connection ends 32 occurs during or after the insertion of the support device 10 into the treatment machine.

In accordance with further embodiments of the present invention, the frame 11 can also be provided with gripping elements 35 configured to allow an operator to grip the support device 10.

According to further embodiments of the invention, moreover, the support device 10 can also comprise an indicator plate attached to the frame 11 and showing graphical signs indicating the positioning modes of the endoscope 13 on the support plane 12. In particular, the graphical signs are configured to reproduce, in scale, the positioning path P of the endoscope 13, to allow the operator to obtain a correct positioning.

The aforementioned graphical signs can also provide indications relating to the start and end positioning points of the endoscope 13 on the support plane 12.

It is clear that modifications and/or additions of parts can be made to the support device 10 for articles as described heretofore, without departing from the field and scope of the present invention.

For example, it can be provided that the first positioning mean 25 is configured to receive and hold the connection terminal 20 in position.

The connection terminal can comprise said first connection ends 31 connected to the feed channels 17 which in this case extend from the connection terminal 20 to the operating terminal 14.

In this case, the treatment liquid is introduced through the connection terminal 20, passes through the feed channels 17 and exits through the operating terminal 14.

It is also clear that, although the present invention has been described with reference to some specific examples, a person of skill in the art shall certainly be able to achieve many other equivalent forms of support device 10, having the characteristics as set forth in the claims and hence all coming within the field of protection defined thereby.

In the following claims, the sole purpose of the references in brackets is to facilitate reading: they must not be considered as restrictive factors with regard to the field of protection claimed in the specific claims.

1. A support device configured to support an article during a washing, sterilization and/or heat disinfection treatment, said article having an oblong and flexible configuration, and being provided with a first terminal part, at least a second terminal part and a flexible connection element connected between said first terminal part and said second terminal part, said device comprising a frame configured to receive and support said article, a first positioning mean attached to said frame and configured to support and hold said first terminal part and a second positioning mean attached to said frame and configured to support and hold said second terminal part, comprising a plurality of holding and positioning elements associated with said frame and defining a plurality of housing seatings disposed aligned along a positioning path and each configured to receive and hold a portion of said flexible connection element in a predefined position.

2. The device as in claim 1, wherein said positioning path has a spiral conformation with a plurality of spirals that wind continuously one inside the other.

3. The device as in claim 1, wherein said housing seatings have a concave conformation and are each provided with an insertion portion open toward the outside and a housing portion, internal with respect to the insertion portion, in which a portion of said article can be positioned, during use, said insertion portion having smaller sizes than those of said housing portion.

4. The device as in claim 1, wherein said frame is defined by a plurality of bars solidly connected to each other, and in that said holding and positioning elements are solidly attached to at least some of said bars.

5. The device as in any claim hereinbefore, characterized in that said first positioning mean is provided with connection devices connectable on one side to devices to feed treatment liquids and on the other side to feed channels provided in said first terminal part.

6. The device as in claim 5, wherein said connection devices comprise at least one connection channel provided with one first connection end and at least a second connection end opposite the first connection end.

7. The device as in claim 6, wherein said first positioning mean is provided with an appendix in which said connection end is provided, said appendix being positioned in correspondence with a lateral edge of said frame.

8. The device as in claim 1, comprising an indicator plate attached to said frame and carrying graphical indication signs of the positioning modes of said article on said support plane.

9. The device as in, claim 1, wherein said first positioning mean defines a first end of said path, said second positioning mean defines a second terminal end of said path and said holding and positioning elements define intermediate segments of said path contained between said first positioning mean and said second positioning mean.

10. A combination of at least one article and a support device in which said article has an oblong and flexible configuration, and is provided with a first terminal part, at least a second terminal part and a flexible connection element connected between said first terminal part and said second terminal part and in which said support device comprises a frame to receive and support said article, a first positioning mean and a second positioning mean both attached to said frame and supporting and holding respectively said first terminal part and said second terminal part, wherein said support device comprises a plurality of holding and positioning elements associated with said frame and defining a plurality of housing seatings disposed aligned along a positioning path and each configured to receive and hold a portion of said flexible connection element in a predefined position.

11. A treatment method for the washing, sterilization and/or heat disinfection of at least one article with a flexible, oblong configuration, and provided with a first terminal part, at least a second terminal part and a flexible connection element connected between said first terminal part and said second terminal part, said method providing to dispose said article on a frame, to support and hold said first terminal part in a first positioning mean and said second terminal part in a second positioning mean, comprising the insertion and holding of portions of said flexible connection element in housing seatings disposed aligned along a positioning path and defined by a plurality of holding and positioning elements associated with said frame.
